(19) United States
(12) Patent Application Publication
(10) Pub. No.: US 2021/0093837 A1
(43) Pub. Date: Apr. 1, 2021

(54) EFFICIENT METHOD AND APPARATUS FOR ACCESSING BLOOD IN FLASH CHAMER OF AN IV CATHETER INSERTION DEVICE

(71) Applicant: William L. SPEARMAN, Beaufort, SC (US)

(72) Inventor: William L. SPEARMAN, Beaufort, SC (US)

(21) Appl. No.: 16/589,602

(22) Filed: Oct. 1, 2019

(51) Int. Cl. A61M 25/06; A61M 25/00

(52) U.S. Cl. CPC A61M 25/0693; A61M 25/0009; A61M 25/0606; A61M 25/0612

(57) ABSTRACT

Disclosed is an apparatus and method for accessing blood located within a flash chamber of an IV catheter insertion device, the apparatus is a separate from the IV catheter insertion device assembly and sized to be attached to a structure of the IV catheter insertion device to secure it thereto (the separate apparatus may be a portion of a protective cover which shields the needle of the device during shipment, a cover which is removed prior to use of the IV catheter insertion device to expose the needle). Once in position, movement of the device from its rearward position to a forward position advances the plunger into contact with the flash chamber plug (or flash filter). Further advancement of the device then advances the flash chamber filter into flash chamber which expresses blood from the flash chamber of the IV catheter insertion device

### CROSS REFERENCE TO RELATED PATENTS

The invention herein described is an improvement to the Flash Chamber Express system as described in patent documents filed by William L. Spearman (and assigned to AV Starr Innovations LLC) in U.S. Pat. Nos. 8,702,658 and 8,728,038 and U.S. Ser. No. 15/444,291 filed Feb. 27, 2017 (the pending patent application U.S. Ser. No. 15/444,291 is sometimes called the Plunger Patent in this document). The specifications and drawings of these patent documents are hereby incorporated by reference into this document.

### BACKGROUND OF THE INVENTION

### Field of Invention

The present invention relates to an IV catheter insertion device having a flash chamber which fills with blood to indicate that the IV catheter has been inserted into the proper location within the vein. In some applications of the insertion device, it is desired to safely access the blood from its flash chamber on demand.

### Background and Background Art

A patient admitted to a hospital, an outpatient treatment center, or an outpatient surgery center will have an intravenous (IV) catheter inserted for easy access for fluids and drugs to be administered to the patient. The IV catheter is typically inserted using an IV catheter insertion device, and some of these IV catheter insertion devices have become known in the industry as JELCOs after the name of one company which brought these insertion devices to market. In addition to placement of an IV catheter, it is frequently necessary for a sample of the patient's blood to be obtained—e.g., for testing, blood typing or other analysis. For many of these blood tests, only a small sample of blood is required. After the IV catheter has been inserted, the healthcare worker may obtain this blood sample by various means. One method would be to have the patient endure another needle stick either by a needle and syringe to draw an aliquot of blood, or by pricking the patient's finger with a lancet for a few drops of blood. Several different types of intravenous (IV) catheter insertion devices currently are on the market. One such IV catheter insertion device is marketed by Smiths Medical under its ProtectIV trademark. This device can be seen represented by U.S. Pat. No. 5,000,740. This device uses a passive system to protect the user from needle stick injury. In its design, this device has a non-removable flash chamber filter. The invention described in this application provides a method and device to allow the user to safely and efficiently access the blood within the flash chamber of these IV insertion devices with a nonremovable flash chamber filter. As patients are treated in the emergency room or prepared for surgery in surgical suites, they often require additional blood testing, such as blood glucose, hemoglobin, pregnancy test, etc. With current point of care (POC) blood testing devices, these test results can be obtained with small volumes of blood, usually one or two drops of blood. Some have proposed to use some of the blood that remains within the JELCO or IV catheter insertion device after the IV catheter has been inserted in a patient for these simple blood tests. Most IV catheter insertion devices include an integrated flash chamber. As the operator inserts the needle of the IV catheter insertion device into the patient's vein, red blood appears within the flash chamber of an IV catheter insertion device, sometimes referred to as a flash of blood. This flash of blood that occurs within the flash chamber shows the operator that the IV catheter has properly entered a vein. The operator watches the flash chamber continuing to fill with blood as the operator advances the IV catheter into the vein, thereby ensuring the IV catheter remains properly positioned within the vein. Usually the proximal end of the flash chamber is blocked by a flash plug. The flash plug typically includes a filter material that allows air to vent from the flash chamber as the blood or fluid fills the chamber, but prevents the blood or fluid from passing from the flash chamber. IV catheter insertion devices of the prior art may have one of two different types of flash plugs. One type of IV catheter insertion device, such as described in U.S. Pat. No. 5,000,740, has a fixed, or non-removable, flash plug. Another type of prior art IV catheter insertion device is seen in U.S. Pat. No. 4,917,671; this type of IV insertion device has a removable flash plug and filter at the proximal end of its flash chamber. When an IV catheter has been placed within the vein, the remainder of the JELCO or IV catheter insertion device is removed from the patient. A small amount of the patient's blood remains within the flash chamber of the JELCO or IV catheter insertion device. By accessing the patient's blood from the flash chamber of the IV catheter insertion device for POC testing, the operator may be able to avoid an additional needle stick to the patient. There are some known approaches to access blood in the flash chamber of an IV catheter insertion device. One IV catheter insertion device, such as U.S. Pat. No. 5,000,740, which is currently marketed by Smiths Medical under its ProtectIV trademark, offers a unique problem when trying to access the blood within the flash chamber since it has a non-removable flash plug. As the flash plug is non-removable, users must adapt special methods if they wish to access the blood within the flash chamber. One such method to access blood in this IV insertion device with a non-removable flash plug involves finding and using an external probe, such as a pen or a golf tee, to push the internal flash plug filter within the flash chamber to expel blood from the flash chamber. The use of an external device such as a golf tee or pen is undesirable for several reasons. The external device must be stored some place that is easily accessible to the operator when the operator is ready to use it. The use of such an external device may be undesirable for safety reasons. If the worker were to reuse any external probe or device (including a pen or a golf tee), cross-contamination may occur from one flash chamber to another, putting other patients and the healthcare worker at risk. In addition the user must align the external probe with the flash plug while pushing the device and flash plug forward within the flash chamber while at the same time keeping the tip of the needle over the correct spot on the testing strip. Spearman patents U.S. Pat. Nos. 8,702,658 and 8,728,038, addressed many of these issues with a device that was built into such an IV catheter device or was attached after-market to provide easy access to the blood within the flash chamber of an IV device with a non-removable flash plug. While these described devices provide easy access to the flash chamber blood in IV devices with non-removable flash plugs, one described device must be included during manufacture of the IV catheter device. The other described device can be attached after market, but must be aligned correctly to the IV catheter insertion device during attachment which, for some operators, makes attachment difficult. The new invention may be used after the IV catheter has been inserted and therefore does not require the manufacturer to include the new invention during production of the IV catheter device. In addition, the new invention is much easier to insert and, by design, aligns easily with the flash chamber filter making for easier attachment by the operator. Prior art, including Spearman patents U.S. Pat. Nos. 8,702,658 and 8,728,038, show a device made of two parts, typically a collar and a plunger. The collar is secured within a section of the IV insertion device (ProtectIV by Smiths Medical) and aligns the plunger with the flash plug filter. The separate plunger moves within the structure of the collar from position one to position two. As the plunger advances, it pushes the flash chamber filter into the flash chamber which expresses the blood within the flash chamber out of the flash chamber through the attached needle to allow the user to drop the blood onto a testing strip. The new device described herein combines the two parts into a single structure. The outer tube of the new device fits within the walls of the finger grip of the ProtectIV device. The fit of the walls of the outer tube aligns the central plunger within the tube with the flash plug filter. The entire device can then be advanced from position one to position two. As the device is advanced, the walls of the outer tube maintain alignment of the attached plunger such that as it is advanced, the tip of the plunger forces the flash plug filter distally into the flash chamber. The advancement of the plunger, and therefore the flash plug filter forces the blood within the flash chamber distally, expressing it from the flash chamber out through the distal tip of the needle from where it can be directed onto a blood testing strip. As the design of the outer tube maintains alignment of the plunger and the flash plug filter, the user does not need to look at the alignment of the distal probe with the filter and can then keep his/her attention on directing the blood onto the testing strip. As the new device can be manufactured as one piece, rather than two separate pieces that must be assembled, the cost of manufacturing is greatly reduced. In addition, as a one-piece design, the new device could also be incorporated into the packaging of an IV insertion device or other similar medical device. For example, every IV catheter comes packaged with a needle cover to protect the needle and prevent accidental needle sticks while handling the device prior to insertion of the IV catheter. This needle cover is usually discarded after uncapping the needle of the IV device. A change to the design of the needle cover could incorporate the single piece device, as will be described herein. This design would not change the function of the needle cover but would add a device to express blood from the used IV catheter insertion device when needed. Indeed, one might also incorporate the design to be included within a cap or other packaging of similar devices such as blood lancets or syringes that could then do double duty as normal caps or covering and a device to express blood from the depicted IV catheter device. The present invention addresses some of these undesirable limitations of prior art to provide an improved device and method for accessing blood from an IV catheter insertion device that has a nonremovable flash plug filter. It is desirable that any part of an IV catheter insertion device (or JELCO) be simple to make. It is also desirable that the device be self-contained and not require additional tools, e.g., an external device like a golf tee or pen, in order to access the blood contained in the flash chamber. If the device is not included during manufacturer of the intravenous catheter insertion device, it is further desirable that the device can be attached to the IV insertion device after the IV catheter is placed and the insertion device has been separated from the IV catheter. The present invention overcomes the disadvantages and limitations of the prior art systems while providing a simple, yet effective, way of accessing the blood contained in the flash chamber of an IV catheter insertion device for testing and analysis without need for a golf tee or ballpoint pen. The present invention overcomes some of the limitations and disadvantages of prior art systems. The present invention provides a way of using an IV catheter insertion device of the prior art while being able to access blood which is located within the flash chamber for testing. The present invention has the advantage that it works with flash plugs which are not removable (for example, as described in U.S. Pat. No. 5,000,740). The present invention improves upon the prior disclosures in the Spearman patents U.S. Pat. Nos. 8,702,658 and 8,728,038. Other objects and advantages of the present invention will be apparent to those of ordinary skill in the art to which the present invention applies in view of the following detailed description of the preferred embodiment taken in conjunction with the accompanying drawings.

### BRIEF DESCRIPTION OF THE DRAWINGS

FIG. 1 (consisting of FIGS. 1A, 1B and 1C) are perspective views of an IV catheter insertion device of the prior art, where FIG. 1A shows the IV insertion device with a needle cover (or safety cap) in place covering the needle (as delivered), FIG. 1B shows the IV insertion device with the needle cover (or safety cap) removed (in preparation for use, prior to placement of the IV catheter) and FIG. 1C shows the IV insertion device of FIGS. 1A and 1B after placement of the IV catheter (the IV catheter within a patient).

FIG. 2 is a perspective view of the invention as a one-piece component with the plunger incorporated into the collar as described within this application.

FIG. 3 (consisting of FIGS. 3A, 3B and 3C) are perspective views of the preferred embodiment of the present invention, as used with the IV insertion device (1C) using the one-piece component depicted in FIG. 2.

FIG. 4 is a perspective view of the improved IV needle cap 150 attached to an IV catheter needle assembly prior to use (prior to being used to place the IV catheter within a patient).

FIG. 5 (consisting of FIGS. 5A, 5B and 5C) is a cross sectional view of a portion of the improved IV needle cap 150 of FIG. 4 and the prior art IV catheter insertion device (after the IV catheter has been removed and installed or inserted within the vein of a patient). 5B and 5C showing the improved needle cap with the cap being inserted adjacent to the flash plug before pushing the flash plug forward to expel blood from the flash chamber.

### DETAILED DESCRIPTION OF THE PREFERRED EMBODIMENT

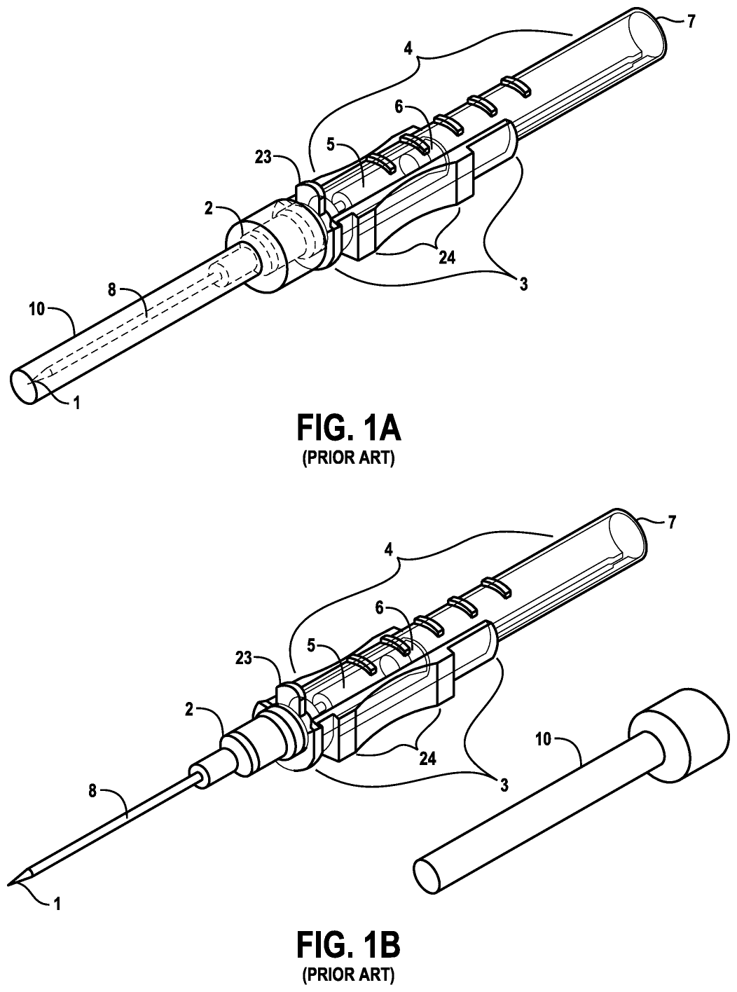

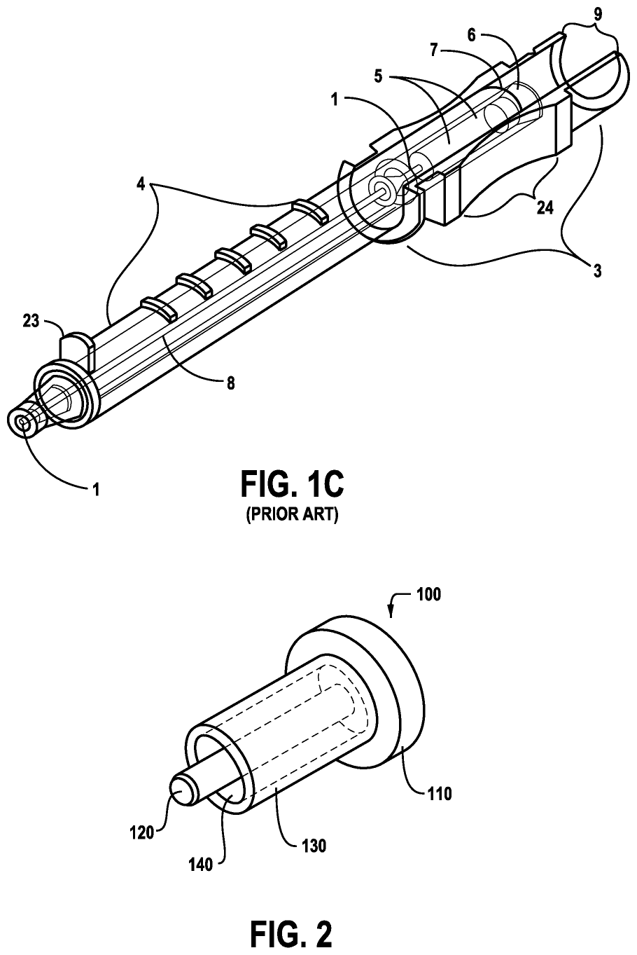

Review of prior art: A review of the workings of an intravenous catheter insertion device such as is sold under the ProtectIV trademark will allow the reader to better understand the present invention. FIG. 1A shows a ProtectIV IV catheter insertion device as delivered with a needle cover 10 provided over the needle 8 which needle 8 terminates in the needle tip 1. Other components shown in FIG. 1A are the IV catheter 2 and a flash chamber 5 and flash plug (or flash filter) 6 mounted within a barrel 4 and having finger grips 24. The barrel 4 has a tubular chamber or opening 9. FIG. 1B shows the catheter insertion device or assembly with its cover 10 removed but prior to placement of the IV catheter 2 in the patient. Using proper technique, the healthcare provider inserts the needle tip 1 through the skin and into the patient's vein (not shown). As with most current IV catheter insertion devices, as the tip 1 of the needle 8 enters the patient's vein, blood flows from the needle tip 1 into the flash chamber 5 where the healthcare provider can confirm access into the vein. The blood continues to flow into the flash chamber 5 as the IV catheter 2 advances into the vein. A flash plug 6 allows air to exit the flash chamber 5 but prevents the blood from exiting from the flash chamber 5. To advance the IV catheter 2 into the vein, the user pushes the finger tab 23 while holding finger grips 24 on main carriage 3. This motion moves the protective tube 4 distally, which slides the IV catheter 2 off the needle and into the vein. At the same time, this motion slides the protective tube 4 distally to cover the needle 8 with its needle tip 1. The protective tube 4 slides through the main carriage 3 and over the needle and needle tip 1 until the tube locks in a second position. For a fuller understanding of the operation of one IV catheter insertion device and its mechanism for protecting the user from inadvertent needle sticks, see the specification and drawings of relevant patents such as U.S. Pat. No. 5,000,740, the specification and drawings of which are hereby incorporated herein by reference. FIG. 1C shows the IV catheter insertion device after the IV catheter has been installed within the patient with the protective tube 4 covering the needle 8 and the flash chamber 5 filled with blood from the patient showing the proper placement of the catheter within the vein.

FIG. 2 illustrates one improvement to the prior art of Spearman to be used with the prior art IV insertion device 1C wherein a device 100 is usable to push the flash plug 6 into the flash chamber 5 and expel blood out of the flash chamber 5 and into the needle 8 and out the needle tip 1 when the plunger 120 is positioned adjacent the flash plug 6 and then advanced into the flash chamber 5.

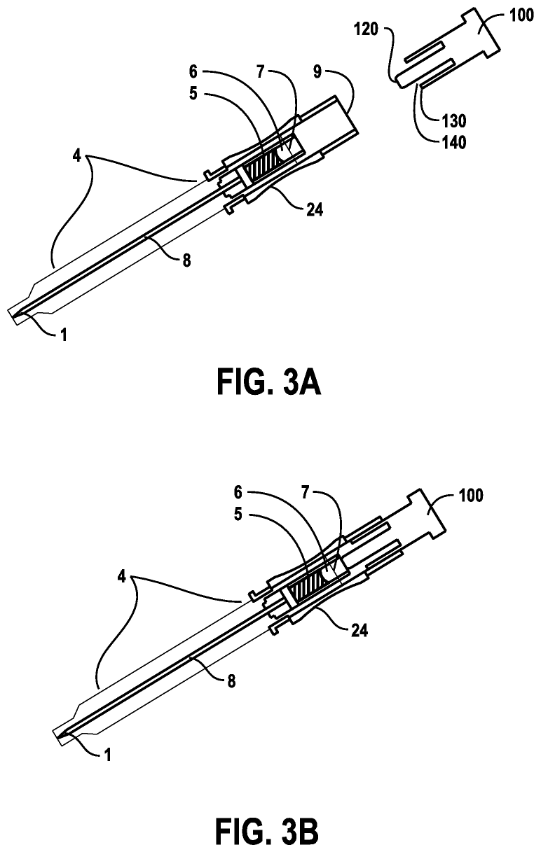

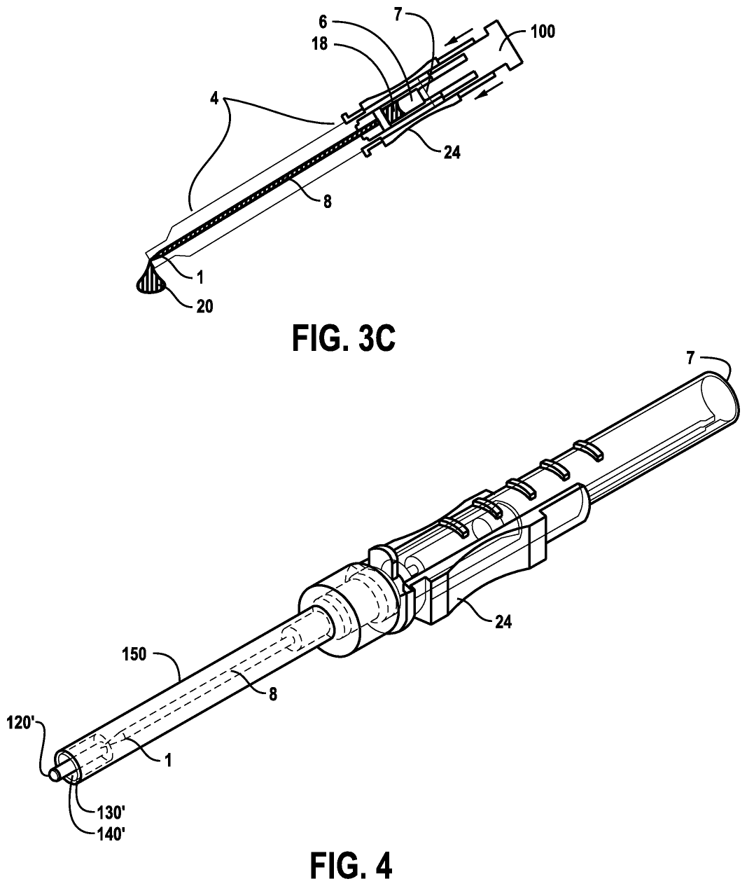

FIG. 3 (consisting of FIGS. 3A, 3B and 3C) illustrates one embodiment of the present invention. The IV catheter is carried on the main carriage 3 and includes finger grips 24 and the flash chamber 5 (shown with blood therein) with the flash plug 6 mounted within the flash chamber 5 which is within the main carriage 3. The diameter of the outer wall of the invention collar 130 fits snugly within the inner walls of the tunnel 9 formed by the finger grips 24 of the IV catheter device (ProtectIV). The invention has a centrally located cylinder (plunger) 120 which may be solid or hollow and have an end portion 120 with an outer diameter that will fit within the inner diameter of the flash chamber 5 of the IV catheter insertion device. The narrow cylinder (the plunger 120) is fixed within the center of the collar cylinder 130. As the collar cylinder 130 is inserted into the finger grip tunnel, the snug fit aligns the device within the center of the finger grip tunnel 9, thereby aligning the plunger 120 with the flash chamber filter (or flash plug) 6. The plunger 120 is of such length, that the plunger 120 does not engage the flash chamber filter 6 until the collar 130 has aligned the plunger to the center of the finger grip tunnel 9. The user can then advance the plunger device from position one (FIG. 3B) to position two (FIG. 3C) by pushing on the proximal end of the device 100. As the device 100 is moved from position one to position two, the plunger 120, by staying in the center of the finger grip tunnel, engages the flash chamber filter 6, pushing it distally within the flash chamber 5. As the flash chamber filter 6 is advanced distally within the flash chamber 5, the blood within the flash chamber 5 is forced distally out of the flash chamber 5 via the needle 8 internal path to the distal tip 1 of the needle 8. As the blood 20 is expressed from the needle tip 1, the user can direct the blood 20 onto a testing strip. After the blood is expressed for testing, the IV insertion device and the invention can be discarded. While one could simply use a small straight cylinder to engage and force the filter distally, the collar aligns the plunger centrally so the user does not need to align the simple plunger.

In the embodiment of FIG. 4, the simple collar/plunger device as describe above could be incorporated into the packaging of certain medical products. For example, the ProtectIV as described, usually has a needle cover 10 to protect the needle 8 and its tip 1 and protect the user from accidental stick prior to insertion of the IV. The invention could be incorporated onto the distal section of the needle cover 10 of FIG. 1A without changing the basic function of the needle cover (the needle cover 150 as shown in FIG. 4, for example), but allowing the user to always have the invention available when starting the IV catheter. The one-piece design of the needle cover 150 in FIG. 4 allows for the structure of the plunger 100 to be made as a part of the manufacturing process of the needle cover without requiring additional pieces or additional manufacturing (or assembly) steps.

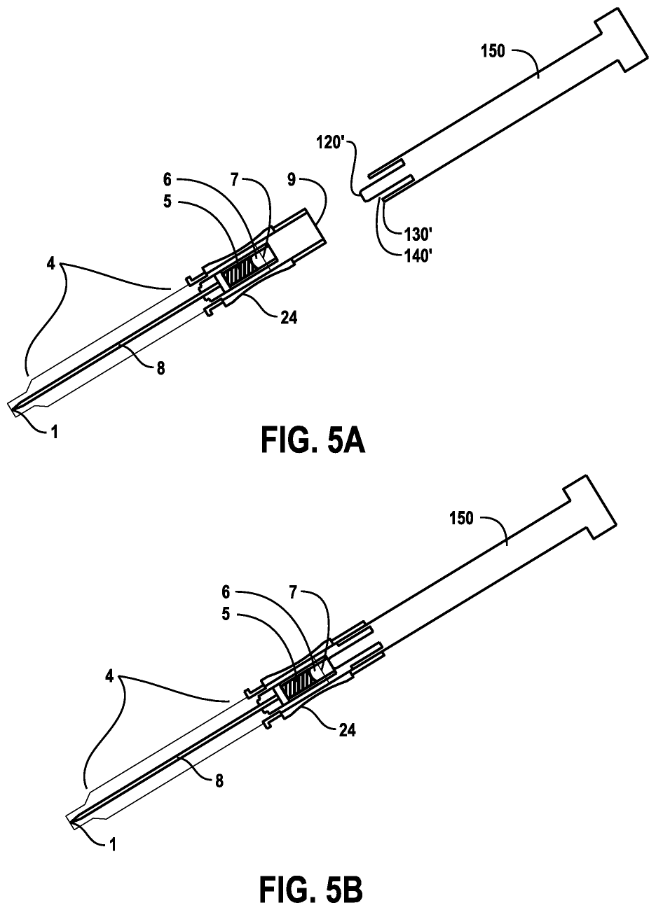

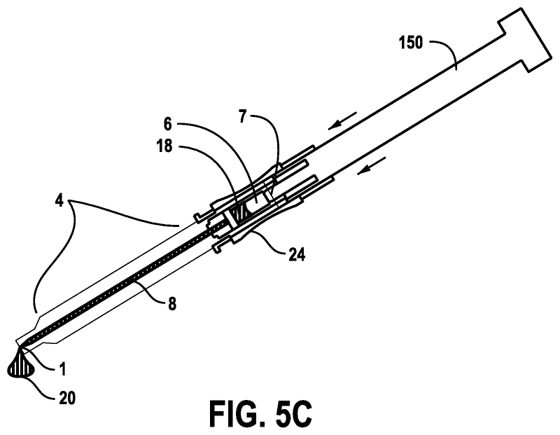

As shown in FIGS. 5A and 5B, the outer walls of the cylinder collar 130 fit snugly within the inner wall of the tunnel 9 formed by the finger grips 24. As the distal end of the cylinder collar 130 enters the finger grip tunnel 9, the snug fit of the collar outer walls within the inner walls of the finger grip tunnel aligns the plunger 120′ centrally into alignment with the flash plug filter 6. Blood 18 is within the flash chamber 5 from the insertion of the IV catheter into the vein.

As shown in FIGS. 5B and 5C, advancement of the modified needle cap 150 from position one (in FIG. 5B) to position two (in FIG. 5C) advances the centrally fixed plunger 120′ to engage the flash plug filter 6 and force the filter 6 distally into the flash chamber 5. The distal advancement of the flash plug filter 6 forces the blood (cross hatched in 5A, 5B and 5C) previously within the flash chamber 5 distally along the needle 8 track and out of the distal tip 1 of the needle 8 where a drop 20 of blood is directed onto a testing strip. In another embodiment, the device could be made with no cylinder collar, simply a central fixed column or plunger. For example, the needle cover 8 could project a simple, thin fixed column at the distal end of the needle cover. The user could direct this plunger to align with the flash chamber filter. Advancing the plunger could then push the flash chamber filter into the flash chamber which would express the sample of blood from the flash chamber via the needle path and onto a test strip. While this embodiment may be more simple to manufacture, the loss of the cylinder collar, as described earlier, forces the user to manually keep the plunger aligned with the flash chamber as the filter is advanced. The centrally fixed plunger with the cylinder collar has the advantage that the collar keeps the centrally fixed plunger properly aligned during advancement of the device. The devices as described made be manufactured of vinyl or plastic material which can be made by injection molds. Similarly, the material may consist any material including, but not limited to wood, metal, glass, or any synthetic, man-made, or natural products.

The present invention may be viewed as a method of manufacturing where a plunger component can be incorporated into a single-piece needle cover and the single-piece needle cover could be manufactured in a single step, such as molding it from a suitable plastic material. Then the user (the healthcare professional) would have at hand a plunger structure merely by removing the protective needle cover as a part of his process for preparing the IV catheter insertion device for use (and would not have to seek a separate part for the function of pressing on the internal flash plug to expel the blood).

Accordingly, the present invention might be seen as a method for using the structure of the improved IV catheter insertion device to provide a tool for expelling blood from the IV catheter insertion device without the necessity for finding a separate tool for pushing on the flash plug to expel blood from the flash chamber.

Of course, many modifications and adaptations of the preferred structure can be made without departing from the spirit of the present invention. For example, the cap structure may have any desired configuration and need not be a particular design or even have symmetry about an axis (in the form a cylindrical structure). Although the cap structure which doubles as a plunger has been described in its preferred embodiment as a single piece of molded plastic, it could be made in a plurality of pieces, if desired, and the insertion device may include additional structure such as a spring to withdraw the needle after it has been used. Additionally, while a molded plastic protective cap is beneficial in some applications, the protective cap could be formed of other materials and/or formed in other manners, if desired, and the plunger portion could be formed as a removable part of the larger protective cap, if desired. Thus it would be appreciated by a man of ordinary skill in the relevant art that the foregoing teaching of the present invention is illustrative of the principles of the present invention and not in limitation of the invention, as the present invention is defined by the claims which follow.

Having thus described the invention, what is claimed is:

1. An intravenous catheter insertion device which allows the user to express blood from a flash chamber of IV catheter insertion device having needle for insertion into a vein and coupled to the flash chamber, a flash plug at the end of the flash chamber, and a needle cover for protecting the needle prior to use, the device comprising a body, a flash chamber and flash plug mounted within the body and a barrel surrounding the flash plug and flash chamber, the needle cover including a plunger formed at its end and sized for insertion into the barrel, allowing movement of the plunger portion of the needle cover to contact the flash plug and move the flash plug into the flash chamber and force blood from the flash chamber out the needle for testing.

2. An intravenous catheter insertion device as described in 1 in which the needle cover with the plunger is manufactured as a single unit and includes a central column which is used to advance the flash plug of an IV catheter insertion device into the flash chamber of the IV catheter insertion device to express blood from the IV catheter flash chamber onto a testing strip.

3. A device as described in 1 wherein the needle cover includes an outer cylindrical collar which fits snugly within the barrel formed by the finger grips of the IV insertion device with the cylinder collar aligns the needle cover centrally within the barrel formed by the finger grips of the IV insertion device with the central alignment of the collar aligns the central plunger with the flash plug and flash chamber of the IV insertion device and maintains alignment of the central plunger with the flash plug and flash chamber as needle cover is moved from a first position to a second position.

4. A device as described in claim 1 wherein the collar is completely cylindrical.

5. A device as described in claim 1 wherein the collar is partially cylindrical.

6. A device as described in 1 wherein the central column or plunger is a solid projection which has an outer diameter that fits within the flash chamber of an IV catheter insertion device and is moved from a first position to a second position to advances the flash plug filter distally into the flash chamber thereby forcing the blood within the flash chamber distally along the needle path out of the distal needle tip and onto a testing strip.

7. A method of making an intravenous catheter insertion device comprising a body including a flash chamber, a flash plug and a housing with an aperture through which the flash plug is movable and a removable cover, with an outer portion of the removable cover being sized to fit within the rear aperture of the housing to move the flash plug from a first position to a second position to expel blood from the flash chamber when the removable cover is removed from its location covering the needle, inserted within the aperture and adjacent the flash plug and moved to move the flash plug from the first position to the second position to expel the blood from the flash chamber.

8. A one-piece device for expressing blood from a flash chamber in an IV catheter insertion device having a tunnel within finger grips, a needle connected to the flash chamber and a movable flash plug defining the flash chamber, the device comprising:
a body having a first size and first shape;
a projecting portion coupled to the body, the projecting plunger portion being smaller than the first size;
wherein the first size and first shape are chosen to fit securely, but movably, within the tunnel and align with the tunnel and the projecting plunger portion is aligned with the movable flash plug and the one-piece device is movable from a first position immediately adjacent to the flash plug to a second position which advances the flash plug to a second position which compresses the flash chamber and expels blood from the flash chamber through the needle.

9. The one-piece device for expressing blood from a flash chamber of claim 8 wherein the IV catheter insertion device has a needle protecting cover and the one-piece device was formed integrally with that cover.

10. A method of making an IV catheter insertion device comprising the steps of forming an insertion device having a housing and including a needle and a flash chamber within
a barrel of the housing, said flash chamber defined by a flash plug; and forming a cover for
covering the needle prior to use, the cover including a portion to fit within the barrel and to compress the flash chamber to expel blood from the flash chamber thru the needle, whereby the cover covering the needle maybe removed and used to compress the flash chamber and express blood from the flash chamber through the needle.

11. A method of using an IV catheter insertion device including a needle, a needle cover and a flash chamber, the method comprising the steps of:
removing the needle covering from the IV catheter insertion device; using the needle to insert the IV catheter within a vein of a patient with blood from the patient
flowing through the needle and into the flash chamber; removing the insertion device and leaving the IV catheter inserted within the patient; and using a portion of the needle cover to compress the flash chamber and to express the blood from the flash chamber through the needle for testing.

12. A method including the steps of claim 11 wherein the portion of the needle cover is sized to fit securely within a barrel of the IV catheter insertion device.

13. A method including the steps of claim 12 wherein the portion of the needle cover includes a portion which is chosen to fit movably within the barrel and a second portion of smaller size which is chosen to push on a flash plug defining the flash chamber to compress the flash chamber to expel blood from the flash chamber.

14. A method comprising the steps of claim 13 wherein the portion of the needle cover chosen to fit within the barrel is slightly smaller than the size of the barrel and the size of the second portion of the needle cover is chosen based on the size of at least one of the flash plug and the flash chamber.
